(19) United States
(12) Patent Application Publication
(10) Pub. No.: US 2009/0216198 A1
(43) Pub. Date: Aug. 27, 2009

(54) SYRINGE NEEDLE GUIDE INSERTION APPARATUS

(76) Inventor: Clifford Salas, Albuquerque, NM (US)

Correspondence Address: ORTIZ & LOPEZ, PLLC, P.O. BOX 4484, ALBUQUERQUE, NM, 87196-4484, US

(21) Appl. No.: 12/038,058

(22) Filed: Feb. 27, 2008

(51) Int. Cl. A61M 5/178

(52) U.S. Cl. 604/187

(57) ABSTRACT

A syringe and needle apparatus, comprising a needle having a sharpened beveled point at one end thereof and having a hollow bore extending from the sharpened end and through the needle to a hub end thereof. The syringe and needle apparatus further includes a syringe including a syringe barrel and a plunger, wherein the barrel possesses a forward hub end adapted to be secured to a hub member of a needle assembly. An identifiable marking is associated with the needle and/or the syringe, such that the identifiable marking ensures a proper and consistent needle position with respect to the sharpened beveled point for entry into a body.

### TECHNICAL FIELD

Embodiments are generally related to medical devices and components. Embodiments are also related to syringes utilized in the delivery of medication to a patient.

### BACKGROUND OF THE INVENTION

A typical hypodermic syringe includes a hollow needle at one end of a cylindrical fluid container and a moveable plunger at the opposite end of the fluid container. The plunger is used to discharge fluid from the fluid container through the needle or to draw fluid through the needle into the cylindrical fluid container. In order that the needle enters painlessly into the skin, the needle tip usually has a small cross-sectional area and an extremely sharp beveled point.

The position of the beveled end is important because it determines how the body will initially react to the introduction of the medication. The beveled end should face down into the surface of the body to minimize the bubbling effect and the pain associated with it. When the beveled end is in a downward position, the bubbling effect just underneath the skin is minimized.

Unfortunately, it is difficult to properly discern the orientation of the needle in a conventional hypodermic syringe and needle device because the cross-sectional area of the needle is very small. This problem is further complicated when needles are used by patients with vision problems such as with the elderly or diabetics in various stages of retinopathy.

### BRIEF SUMMARY

The following summary of the invention is provided to facilitate an understanding of some of the innovative features unique to the present invention and is not intended to be a full description. A full appreciation of the various aspects of the invention can be gained by taking the entire specification, claims, drawings and abstract as a whole.

It is therefore one aspect of the present invention to provide for an improved medical device.

It is another aspect of the present invention to provide for an improved hypodermic syringe and needle device for the delivery of medication to a patient.

It is a further aspect of the present invention to provide for a syringe insertion guide for the efficient delivery of medication to a patient.

It is further aspect of the present invention to provide for a syringe insertion guide that will properly orient the position of the beveled end of the needle to reduce the pain associated with the introduction of medication into the body.

The above and other aspects of the invention can be achieved as will now be briefly described. A medical device is disclosed, which is generally composed of a syringe and needle apparatus. The needle can be configured to include a sharpened beveled point at one end and a hollow bore extending from the sharpened end and through the needle to a hub end. A syringe assembly generally includes a syringe barrel and a plunger. The syringe barrel has a forward hub end adapted to be secured to the hub member of the part containing the needle. An identifiable marking can be placed on the shaft of the needle and/or as well on the syringe barrel, depending upon design considerations. Such identifiable markings ensure a proper and consistent needle position with respect to the sharpened beveled point for entry into the body.

The identifiable marking(s) can be visible markings and/or that is identifiable to the touch. The syringe and needle apparatus can be connected to one another permanently or the needle may be replaced with respect to the syringe in order to provide for a reusable syringe device. Alternatively, the entire syringe and needle apparatus can be a completely disposable unit. In an alternative embodiment, the syringe and needle apparatus can be configured with an axially locking key mechanism to secure the needle assembly to the syringe barrel. Further, the syringe and needle apparatus comprises a screw-on type connection to secure the needle assembly to the syringe barrel. Other securing mechanisms can be used to properly secure the needle assembly to the syringe barrel to ensure a proper fit and proper alignment of the needle.

### BRIEF DESCRIPTION OF THE DRAWINGS

The accompanying figures, in which like reference numerals refer to identical or functionally similar elements throughout the separate views and which are incorporated in and form part of the specification, further illustrate embodiments of the present invention.

FIG. 1 illustrates a syringe and needle apparatus from a side view, in accordance with a preferred embodiment;

FIG. 2 illustrates the syringe and needle apparatus depicted in FIG. 1 from a top view, in accordance with a preferred embodiment;

FIG. 3 illustrates a syringe and needle apparatus from the side view, in accordance with an alternative embodiment;

FIG. 4 illustrates a syringe and needle apparatus of FIG. 3 from a tope view, in accordance with an alternative embodiment;

FIGS. 5 and 6 illustrate a key locking mechanism that can be adapted for use with a syringe barrel assembly and needle assembly, in accordance with an alternative embodiment;

FIG. 5 illustrates the top view of a syringe barrel assembly separated from a needle assembly, and an identifiable marking along a surface of a syringe barrel, in accordance with an alternative embodiment;

FIG. 6 illustrates a side view a raised molding that runs axially along a syringe barrel, wherein the molding has a flat surface to allow for additional markings, in accordance with an alternative embodiment;

FIG. 7. Illustrates an axial view of a key locking mechanism of a syringe barrel assembly and a needle assembly, in accordance with an alternative embodiment;

FIG. 8 illustrates a close up view of a needle assembly and a marking along a shaft of a needle, in accordance with an alternative embodiment;

FIG. 9 illustrates a top view of a screw-on syringe and needle apparatus, in accordance with an alternative embodiment; and

FIG. 10 illustrates an axial view of a molded flange near an end of the barrel of a syringe assembly toward a plunger, in accordance with an alternative embodiment.

### DETAILED DESCRIPTION OF THE INVENTION

The particular values and configurations discussed in these non-limiting examples can be varied and are cited merely to illustrate embodiments of the present invention and are not intended to limit the scope thereof.

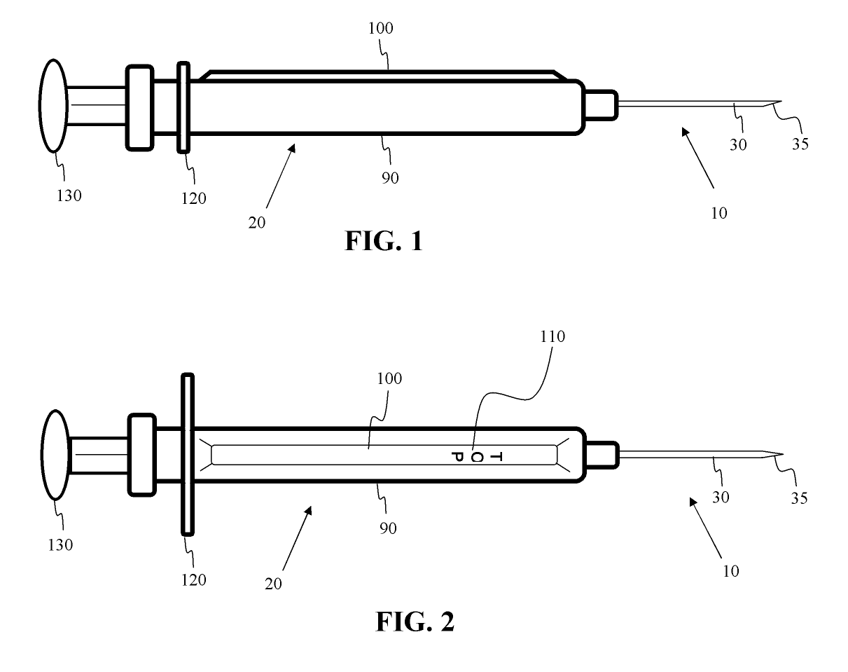

FIG. 1 illustrates a side view of a syringe and needle apparatus 10 that includes a syringe 20 in association with a needle 30, in accordance with a preferred embodiment. The needle 30 includes a sharpened beveled point 35 at one end. The apparatus 10 further includes two other components, including a syringe barrel 90 and a plunger 130. A raised molding 100 is also depicted in FIG. 1 with respect to the syringe barrel 90. A molded flange 120 can be positioned near the end of the syringe barrel assembly 20 toward the plunger 130 to assist a user in operating the apparatus 10.

FIG. 2 illustrates a top view of the syringe and needle apparatus 10 of FIG. 1, in accordance with a preferred embodiment. Note that in FIGS. 1-10, identical parts or elements are generally indicated by identical reference numerals, although specific figures among FIGS. 1-10 may illustrate different embodiments of the present invention. As depicted in FIG. 2, an identifiable marking 110 can be placed on a raised molding 100 of the syringe barrel 90. Note that in the particular embodiment depicted in FIGS. 1-2, the needle 30 can constitute a replaceable/disposable needle with respect to the syringe barrel 90 and syringe 20, or may be permanently connected to the syringe barrel 90/syringe 20, depending upon design considerations. Apparatus 10 may also constitute a disposable device, again depending upon design considerations. The identifiable marking 110 thus functions a syringe needle guide apparatus that permits a user of apparatus 10 to properly discern the orientation of the needle 30 with respect to a point on a human body for injection into the body.

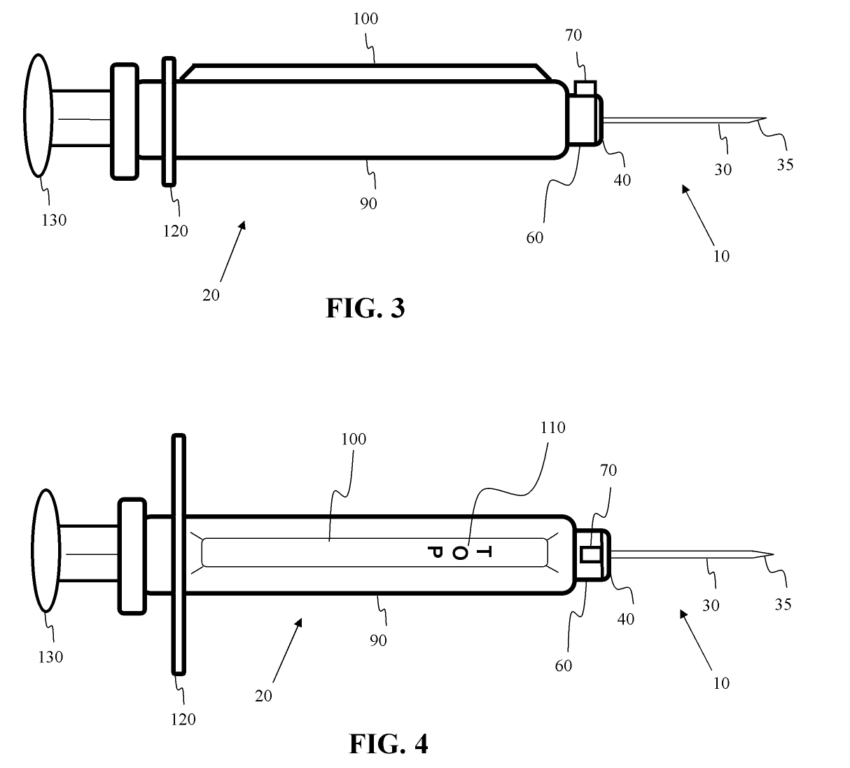

FIG. 3 illustrates a side view of a modified version of apparatus 10, including syringe 20 and needle 10, in accordance with an alternative embodiment. In the configuration depicted in FIG. 3, needle 30 can be configured to include a sharpened beveled point 35 at one end and a hollow bore extending from the sharpened point 35 and through the needle to a hub end 40. The syringe apparatus 10 depicted in FIG. 3 generally includes two parts, the syringe barrel 90 and the plunger 130. The syringe barrel 90 further can be configured to include a forward hub end 60 adapted to be secured to a hub member 40 of the part containing the needle 10.

FIG. 4 illustrates a top view of the syringe and needle apparatus 10 depicted in FIG. 3, in accordance with an alternative embodiment. As depicted in FIG. 4, the identifiable marking 110 can be placed on a raised molding 100 of the syringe barrel 90. The identifiable marking 110 depicted in FIG. 4 can ensure a proper and consistent needle position with respect to the sharpened beveled point 35 for entry into the body.

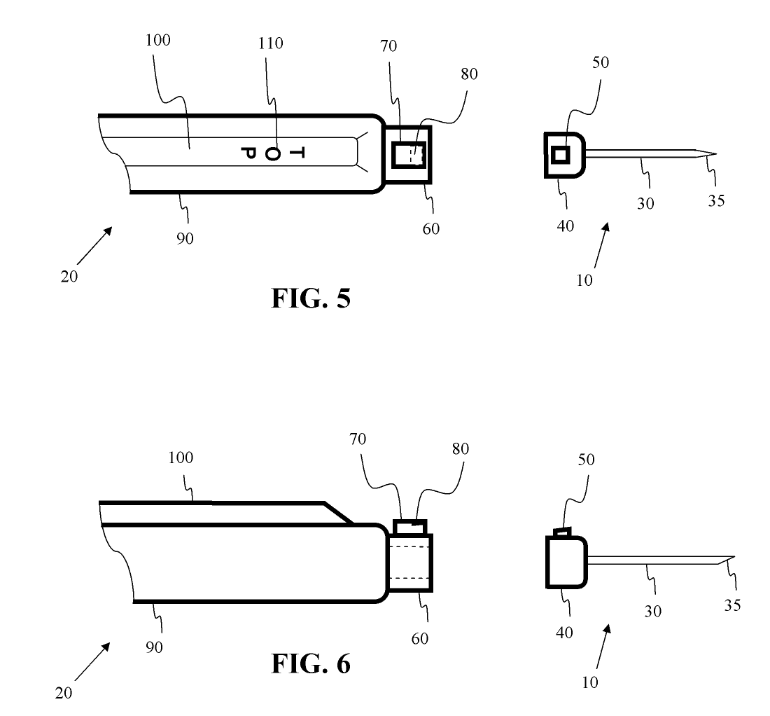

FIG. 5 illustrates the syringe barrel assembly 20 separated from the needle assembly 10, in accordance with an alternative embodiment. FIG. 5 further illustrates an identifiable marking 110 along the surface of the syringe barrel 90. The marking 110 can be annotated with a phrase or word such as, for example, “TOP” but can be written to include another marking that properly communicates the upward position of the syringe for a user.

FIG. 6 illustrates a side view of the syringe barrel assembly and needle assembly 10 depicted in FIG. 5, in accordance with an alternative embodiment. FIG. 6 further illustrates a configuration in which the raised molding 100 runs axially along the syringe barrel 90. The molding 100 can be modified to further include a flat surface to allow for additional markings. The needle assembly 10 can be modified to include a raised notch 50 on top of the needle hub 40. In the particular embodiment depicted in FIG. 5, the raised notch 50 increases in height in a ramping shape. Additionally on the syringe hub 60, a lock housing 70 can be provided with a lip 80 to secure the raised notch 50 on the needle hub 40. The raise notch 50 and/or molding 80 can thus function as a syringe needle guide apparatus. The key locking mechanism 50, 70, and 80 can ensure a secure and properly oriented fit when the syringe barrel assembly 20 connects to the needle assembly 10. This key locking mechanism 50, 70, and 80 is one example of a securing means for the syringe barrel assembly 20 and the needle assembly 10 and is not intended to limit the scope of such a securing means.

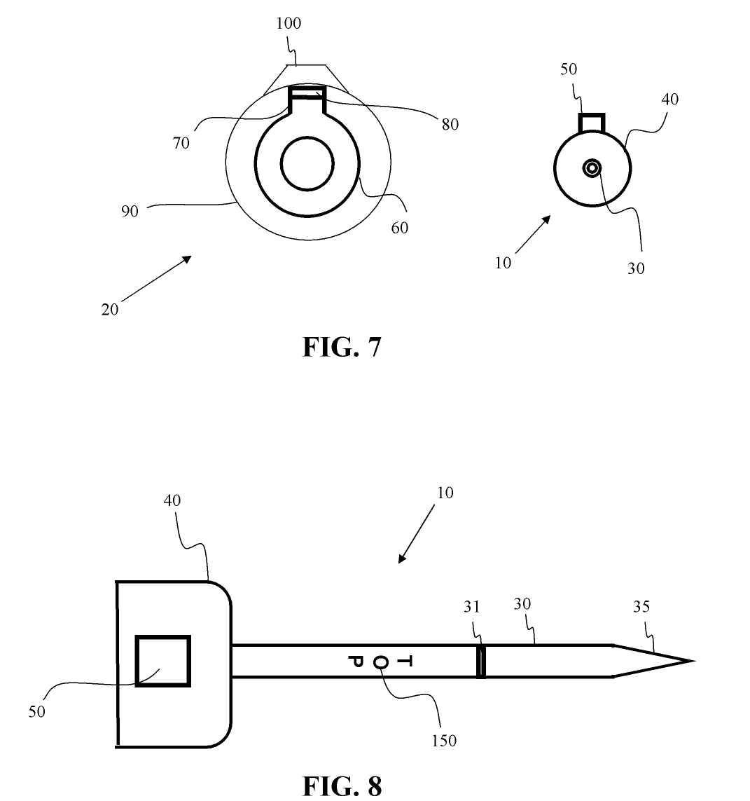

FIG. 7 illustrates an axial view of the key locking mechanism 50, 70, and 80 of the syringe barrel assembly 20 and the needle assembly 10, in accordance with an alternative embodiment. FIG. 7 also illustrates a flat surface of the raised molding 100 of the syringe barrel 90.

FIG. 8 illustrates a close up view of the needle assembly 10 and a marking 150 along the shaft of the needle 30, in accordance with an alternative embodiment. The marking 150 can be located on the top side which is opposite that of the opening of the beveled end 35 of the needle 30. The marking 150 can be annotated with the words “TOP”, but can be any other marking to communicate the upward position of the syringe. Note that in addition to or in lieu of the marking 150, a laser marking 31 may be provided on the top of the needle 30 or along a shaft of the needle 30, but preferably on top of the needle 30. The laser marking 31 thus may assist the user in properly placing the needle 30 with respect to a human body for injection of the needle 30 into the body. Laser marking 31 and/or marking 150 thus function as a laser guide insertion apparatus for properly guiding entry of the needle 30 into a human body by a user.

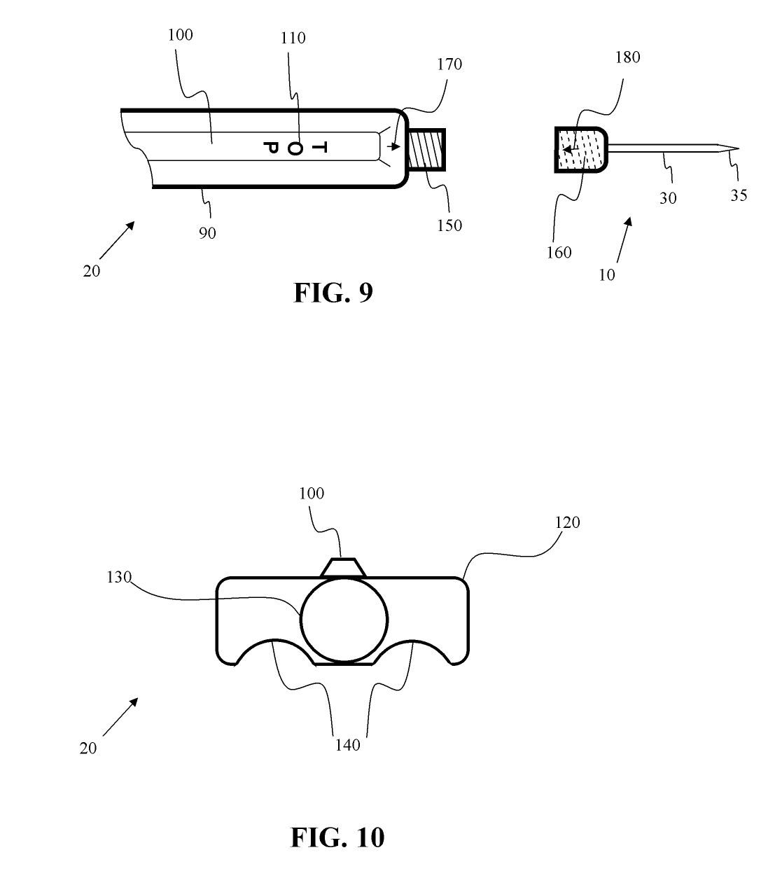

FIG. 9 illustrates the top view of a screw-on syringe and needle apparatus, in accordance with an alternative embodiment. The hub of the syringe barrel has screw grooves 150 on the outside of the hub. The hub of the needle has screw grooves 160 on the inside of the hub. The screw grooves 150 of the barrel and the screw grooves 160 of the needle allow for a secure fit when screwed together. A marking 170 on the hub of the syringe barrel and a marking 180 on the hub of the needle can be provided to ensure proper alignment of the needle to the syringe barrel.

FIG. 10 illustrates the axial view of the molded flange 120 near the end of the syringe barrel assembly 20 toward a plunger 130, in accordance with an alternative embodiment. As depicted in FIG. 10, there are two opposite sides for grip. The flange 120 can be provided with curved indentations 140 on the bottom sides thereof for alignment with fingers by a user. The location of the curved indentations 140 can also aid the user to ensure proper orientation of the needle as it enters the body.

Based on the foregoing, it can be appreciated that prior art hypodermic syringes do not possess a distinguishing feature that can assist a user in properly orienting the needle thereof to ensure a minimal pain associated with delivering medicine into the body. In order for the needle to painlessly enter into the skin, the needle tip usually has a small cross-sectional area and an extremely sharp beveled point. The position of the beveled end is important because it determines how the body will initially react to the introduction of the medication. The beveled end should face down into the surface of the body to minimize the bubbling effect and the pain associated with it. When the beveled end is in a downward position, the bubbling effect just underneath the skin is minimized. Unfortunately, in prior art situations, it is difficult to properly discern the orientation of the needle in a conventional hypodermic syringe because the cross-sectional area of the needle is very small. This problem is further complicated when needles are used by patients with vision problems such as with the elderly or diabetics in various stages of retinopathy.

The disclosed embodiments therefore offer several distinguishing features that properly orient the needle as it enters the body. The unique shape of the syringe can include a flat surface along the top of the syringe barrel, a curved flange for grip, and visible markings to assist the user in ensuring that the beveled end of the needle is face-down as it enters the body. This result in a smooth entry of the needle into the body and minimal pain associated with the delivery of the medicine thereafter.

It will be appreciated that variations of the above-disclosed and other features and functions, or alternatives thereof may be desirably combined into many other different systems or applications. Also that various presently unforeseen or unanticipated alternatives, modifications, variations or improvements therein may be subsequently made by those skilled in the art which are also intended to be encompassed by the following claims.

1. A syringe and needle apparatus, comprising:
a needle having a sharpened beveled point at one end thereof and having a hollow bore extending from said sharpened end and through said needle to a hub end thereof;
a syringe including a syringe barrel and a plunger, said barrel having a forward hub end adapted to be secured to a hub member of a needle assembly; and
an identifiable marking associated with said needle and/or said syringe, wherein said identifiable marking is indicative of the position of said needle and ensures a proper and consistent needle position with respect to said sharpened beveled point for entry into a body.

2. The apparatus of claim 1 wherein said identifiable marking is located along a shaft of said needle and comprises language or symbols indicative of an identifiable direction.

3. The apparatus of claim 1 wherein said identifiable marking is identifiable by a user touch via a raised notch located on an external surface of said hub end.

4. The apparatus of claim 1 wherein said identifiable marking is located along a shaft of said syringe barrel and comprises language or symbols indicative of an identifiable direction.

5. The apparatus of claim 1 wherein said identifiable marking is identifiable by a user touch via a raised molding on said syringe barrel.

6. The apparatus of claim 1 wherein said identifiable marking comprises a distinguishable shape to the touch of said syringe barrel for proper orientation of said syringe for entry of said needle into said body.

7. The apparatus of claim 1 wherein said identifiable marking comprises an identifiable laser marking.

8. The apparatus of claim 1 wherein said hub of syringe is marked with said identifiable marking, said identifiable marking comprising an identifiable painted marking.

9. The apparatus of claim 1 wherein said syringe barrel of syringe is marked with said identifiable marking, said identifiable marking comprising an identifiable painted marking.

10. The apparatus of claim 6 wherein said distinguishable shape of said barrel includes a flat surface axially along one side thereof.

11. The apparatus of claim 6 wherein said distinguishable shape of said barrel includes a raised surface identifiable to a touch by said user.

12. The apparatus of claim 6 wherein said distinguishable shape of said barrel includes a molded flange near an end of said barrel on two opposite sides thereof for grip, said molded flange having a curved indentation on a bottom side for alignment with fingers by a user for the purpose of identifying the orientation of said needle as it enters a target.

13. The apparatus of claim 1 wherein said identifiable markings and said distinguishable shapes are placed on opposite side of said beveled opening of said needle.

14. The apparatus of claim 1 wherein said syringe and said needle are permanently connected as a single unit.

15. The apparatus of claim 1 further comprising: a hub member located between said syringe barrel and said needle and interconnecting said syringe barrel and said needle, said hub member including an axially locking mechanism therein in communication with a needle matching locking mechanism.

16. The apparatus of claim 15, wherein said locking mechanism of said hub member is in communication with said needle, comprises:
a ramp shaped raised notch on the surface of the needle hub; and
a raised locking housing for said ramp shaped raised notch on said hub of barrel, wherein said ramp shaped raised notch and raised locking housing communicate securely and are secured in place by a clamping effect, said ramp shaped raised notch and raised locking housing placed on the opposite side of said bevel.

17. A syringe and needle apparatus, comprising:
a needle having a sharpened beveled point at one end thereof and having a hollow bore extending from said sharpened end and through said needle to a hub end thereof;
a syringe including a syringe barrel and a plunger, said barrel having a forward hub end adapted to be secured to a hub member of a needle assembly; and
an identifiable marking associated with said needle and/or said syringe, wherein said identifiable marking is located along a shaft of said needle and/or a shaft of said syringe, wherein said identifiable marking is indicative of the position of said needle and ensures a proper and consistent needle position with respect to said sharpened beveled point for entry into a body.

18. The apparatus of claim 17 wherein said identifiable marking comprises a distinguishable shape to the touch for proper orientation of said syringe for entry of said needle into said body.

19. The apparatus of claim 17 wherein said identifiable marking comprises an identifiable laser marking, wherein said identifiable laser marking comprises language or symbols indicative of an identifiable direction.

20. A syringe and needle apparatus, comprising:
a needle having a sharpened beveled point at one end thereof and having a hollow bore extending from said sharpened end and through said needle to a hub end thereof;
a syringe including a syringe barrel and a plunger, said barrel having a forward hub end adapted to be secured to a hub member of a needle assembly; and
an identifiable marking associated with said needle, said identifiable marking comprising a laser marking located on said needle, wherein said identifiable marking comprises language or symbols indicative of an identifiable direction and ensures a proper and consistent needle position with respect to said sharpened beveled point for entry of said needle into a body.
